(19) United States
(12) Patent Application Publication
(10) Pub. No.: US 2005/0268274 A1
(43) Pub. Date: Dec. 1, 2005

(54) Wet-laid tissue sheet having an air-laid outer surface

(76) Inventors: Paul Douglas Beuther, Neenah, WI (US); Frank Gerald Druecke, Oshkosh, WI (US); Margaret Katherine Huseman, Cincinnati, OH (US); Kambiz Bayat Makoui, Neenah, WI (US)

Correspondence Address: KIMBERLY-CLARK WORLDWIDE, INC., 401 NORTH LAKE STREET, NEENAH, WI, 54956, US

(21) Appl. No.: 10/857,124

(22) Filed: May 28, 2004

(51) Int. Cl. A61F 13/15; A61F 13/20

(52) U.S. Cl. 717/100

(57) ABSTRACT

An air-laid web is combined with a wet-laid web prior to drying to form a layered tissue sheet. The resulting sheet has a unique combination of properties, which can include greater bulk and fuzziness on the air-laid side and greater strength and more smoothness on the wet-laid side.

### BACKGROUND OF THE INVENTION

Wet-laid tissues have been made commercially for a long time. Conventional wet-laying processes generally involve depositing an aqueous suspension of papermaking fibers onto a forming fabric to form a web, transferring the web to a drying section and drying the web. Prior to drying, the web is typically dewatered by vacuum or pressure to a consistency of about 30 percent. The web can be dried by throughdrying or by using a Yankee dryer. The resulting dried web may be creped or left uncreped. The properties of wet-laid tissue sheets can vary greatly depending upon the process configuration and the fibers being used. In general, wet-laid processes are fast, reliable and reasonably cost-effective.

Also known in the art is the use of air-laid fibers for making tissue products, although commercial tissue processes are not common. Air-laid processes offer the potential for producing very soft (high surface fuzziness) products with high bulk because of the low fiber-to-fiber hydrogen bonding that characterizes the air-laying process. By way of example, an air-laid tissue making process was briefly commercialized by Kimberly-Clark Corporation in the early 1980's with the production of Kleenex® Softique® facial tissue. For that process, debonded northern softwood kraft pulp fibers were formed into an air-laid web and bonded together using two high pressure rolls to emboss the web. A dry strength resin was printed onto the web surface to increase strength and decrease lint without significantly decreasing the sheet softness. However, the process was discontinued shortly after production started, due in part to slow machine speeds, high costs, and difficulties in maintaining the desired sheet formation quality.

Therefore there is a need for a wet-laid tissue making process that can provide the usual advantages associated with wet-laying processes and also provide the product advantages of softness and bulk associated with air-laying processes.

### SUMMARY OF THE INVENTION

It has now been discovered that a tissue sheet with unique properties can be produced by providing a layered structure in which one outer layer of the sheet is formed by air-laying and the other outer layer is formed by wet-laying. The resulting tissue sheet is smooth and strong on one side (the wet-laid side) and very soft and fuzzy on the other side (the air-laid side). In addition, because the air-laid fibers are combined with wet-laid fibers in a wet-laying process, spraying binders onto the sheet to maintain sheet integrity is not necessary, thereby eliminating a machine speed limitation. Also, by combining the air-laid fibers with the wet-laid web prior to drying, the consistency of the combined web is immediately reduced, thereby correspondingly reducing the drying load in the drying section of the overall process.

Hence in one aspect, the invention resides in a layered tissue sheet having two outer surfaces, said sheet comprising: (a) an air-laid outer layer of papermaking fibers which provides one outer surface of the sheet; and (b) a wet-laid outer layer of papermaking fibers which provides the second outer surface of the sheet, wherein the air-laid outer surface is relatively fuzzy compared to the wet-laid outer surface. In most cases, the air-laid outer layer is also relatively less dense than the wet-laid outer layer. The relative density difference between the air-laid outer layer and the wet-laid outer layer can be observed by cross-sectional photographs of the tissue sheet. The relative fuzziness of the air-laid outer layer and the wet-laid outer layer can be determined by qualitative or quantitative methods hereinafter described. As used herein, the term “sheet” is synonymous with a “single ply”. It is a component of a final tissue “product”, which may consist of one or more sheets.

In another aspect, the invention resides in a multi-ply tissue product having two outer plies, wherein each of the two outer plies is a layered tissue sheet as described above. The outer plies can be oriented such that both air-laid outer surfaces are outwardly-facing, or such that both air-laid surfaces are inwardly-facing, or such that one air-laid surface is outwardly-facing and the other air-laid surface is inwardly-facing. If both air-laid surfaces are oriented to be outwardly-facing, the resulting tissue product will feel very soft on both sides. If both air-laid surfaces are inwardly-facing, the resulting tissue product will feel very smooth on both sides, yet will still have high bulk. If only one air-laid surface is oriented as an outwardly-facing surface, then the resulting tissue product will have two very different sides, one being smooth and the other being fuzzy. Of course, the tissue sheets of this invention can also be used as a single-ply tissue product.

In another aspect the invention resides in a method of making a unitary layered tissue sheet comprising: (a) forming a wet-laid web by depositing an aqueous suspension of papermaking fibers onto a forming fabric to form a wet web; (b) forming an air-laid web by depositing an airborne suspension of papermaking fibers onto a forming fabric; (c) combining the air-laid web with the wet web to form a layered web having a wet-laid layer and an air-laid layer; (d) drying the layered web; and (e) optionally creping the dried web.

In another aspect the invention resides in a method of modifying a wet-press tissue machine comprising a headbox for depositing an aqueous suspension of papermaking fibers onto a forming fabric to form a wet-laid web, a drying section for drying the wet-laid web, and a transfer section in which the wet-laid web is supported by a fabric or felt and transferred to the drying section, wherein the tissue machine is modified by adding an air former for depositing an air-laid web of papermaking fibers and positioning the air former such that the air-laid web of fibers is combined with the wet-laid web prior to the drying section.

The fuzziness of the tissue sheets of this invention can be qualitatively observed by feel or by viewing the surfaces of the sheets through a microscope. Quantitatively, the fuzziness can be measured by the Fuzz-On-Edge (FOE) test, hereinafter defined. The FOE value of the air-laid outer surface of the tissue sheets of this invention is greater than the FOE value of the wet-laid outer surface of the sheet. In addition, the difference in FOE values between the two outer surfaces can be about 2.00 or greater, more specifically about 2.50 or greater, more specifically about 3.00 or greater, more specifically from about 2.00 to about 6.00, still more specifically from about 2.50 to about 5.00, still more specifically from about 3.00 to about 4.00. By comparison, the FOE values of some commercially-available wet-laid tissue products are usually less than 1 on both sides, and almost always less than 2. Examples of tissue sheets with FOE values greater than 2.0, such as those disclosed in U.S. Pat. No. 6,585,855, would have a high FOE value on both sides of the tissue sheet.

The sheet bulk (hereinafter defined) of the tissue sheets of this invention, measured as produced by the tissue machine prior to any post-treatment, such as calendaring, or as measured in the final tissue product, can be from about 7 to about 15 cubic centimeters per gram (cc/g), more specifically from about 9 to about 15 cc/g and still more specifically from about 10 to about 14 cc/g. The high sheet bulk values are primarily attributable to the relatively low density of the air-laid layer of the sheet.

In general, the basis weight of each layer can be about equal. More specifically, the ratio of the dry fiber basis weight of the air-laid layer relative to that of the wet-laid layer can be from about 0.25 to about 4, more specifically from about 0.3 to about 3, more specifically from about 0.5 to 2, and still more specifically from about 0.75 to about 1.25. In absolute terms, the basis weight of the air-laid layer and the wet-laid layer can each be, independently of each other, from about 3 to about 50 grams per square meter (gsm), more specifically from about 5 to about 40 gsm and still more specifically from about 7 to about 30 gsm.

In carrying out the method of this invention, a number of different options are available. In one embodiment, the layered web is dried and creped by adhering the wet-laid layer side of the web to the surface of a Yankee dryer. In another embodiment, the wet-laid web is formed on a crescent former, in which the aqueous suspension of papermaking fibers is injected into a forming zone created by the convergence of a forming fabric and a felt, wherein the air-laid web is transferred from the air-laying forming fabric to the felt prior to the formation of the wet-laid web. In another embodiment, the air-laid web is combined with the wet web after the wet web is formed.

Machine speeds can be those typically used for wet-laid tissue machines. Such speeds can be about 2000 feet per minute (fpm) or greater, more specifically from about 2500 to about 4000 fpm or greater. While any suitable air-laid former can be used for purposes herein, many air-laid formers can be speed-limited. An improved air-laid former design would be a headbox-style former that blows air and fibers nearly parallel to the forming fabric with vacuum assist underneath, similar to a Fourdriner machine or a twin-wire former. The “slice” opening of the former would be about 25-100 mm high, with a forming zone of about 0.5-3 meters long. The forming vacuum would be about 6 inches of water (1.5 kiloPascals) or less. It is anticipated that lower pressures could be used with appropriate forming fabric selection. Once formed, the fibers would be held in place on the fabric with continuous vacuum or by being sandwiched between two fabrics until transported to the location where they are combined (couched) with the wet-laid web. This would serve to prevent disruption of the unbonded or weakly-bonded fibers by the boundary layer air. If the air-laid fibers remain on the outside of the resulting layered web after couching, it is advantageous to keep them sandwiched between two fabrics or under continuous vacuum until pressed to the Yankee dryer. Alternatively, bonding agents could be applied to the air-laid fibers to keep them in place prior to drying, as well as to aid in final product strength development and minimization of lint when the air-laid fibers on the outside of the product.

Fibers useful for purposes of this invention include any natural or synthetic papermaking fibers useful for making facial tissue, bath tissue, paper towels, table napkins and the like. For the wet-laid layer, hardwood fibers or furnishes consisting primarily of hardwood fibers are particularly suitable in order to provide the greatest amount of softness. Chemical strength agents can also be added to the furnish, which is conventional for wet-laid tissue products. For the air-laid layer, softwood fibers or furnishes consisting primarily of softwood fibers are particularly suitable in order to provide high bulk. Fibers for the air-laid layer can also include fibers with a chemical additive such as lotion(s) and/or herbal extracts in order to provide additional benefits. Layer integrity can be imparted through the use of thermoplastic synthetic fibers, followed by heating to tackify the surface of the thermoplastic fibers, thereby creating bond sites. Particularly suitable thermoplastic synthetic fibers include those having a sheath/core structure. A specific commercially available fiber is KoSa T255 bicomponent fiber having a polyester core and a polyethylene terephthalate/polyethylene copolymer sheath, or Fibervisions AL-Adhesion-C.

Layer integrity for the air-laid layer, if deemed necessary, can also be provided by spraying chemical bonding agents onto the surface of the newly-formed air-laid web. Suitable bonding agents include cationic, non-crosslinking binders that have a low glass temperature (Tg). Some examples of these are Carboset 519 and Crepetrol A6115 (Hercules Chemical Co.). The latter compound is actually a creping resin but it can act as a strengthening agent as well. Also suitable is Airflex 410 made by Air Products, which is an ethylene vinylacetate copolymer. Certain cross-linking binders may also be useful.

Although the tissue sheets of this invention are particularly suited for two-ply products, the tissue sheets of this invention can also be used as a single-ply product having a high level of strength, softness, and fuzziness. The wet-laid side of the sheet would provide high strength, softness and fuzziness in the manner already practiced for conventional wet-pressed, two-ply tissue. More specifically, the wet-laid side of the combined tissue web would be the side attached to the Yankee dryer and subsequently creped. The fiber mixture for the wet-laid side of the web could be combinations of long and short fibers and also could be either blended or layered. The other side of the sheet would be the air-laid web and would be the “air side” of the sheet (not adhered to the Yankee dryer surface). The air-laid layer of the resulting sheet would provide high softness and high fuzziness properties. Such a product could provide the strength and softness of current two-ply tissues and greater bulk than conventional wet-pressed tissue. One particular one-ply product structure envisioned is a sheet having about 60 percent of the total basis weight being wet-laid fibers formed with a crescent former configuration and about 40 percent of the total basis weight being air-laid fibers. The finished basis weight would be in the 25 to 32 gsm range. The air-laid fibers would be deposited onto the already-formed wet-laid structure at some point after typical vacuum dewatering and before attachment to the Yankee dryer. The resulting layered web would be attached to the Yankee dryer by means of a carrying fabric and through the use of appropriate dryer coating chemical mixtures to achieve desired sheet adhesion and creping intensity.

As used herein, “sheet bulk” is calculated as the quotient of the “caliper” (hereinafter defined) of a tissue sheet, expressed in microns, divided by the dry basis weight, expressed in grams per square meter. The resulting sheet bulk is expressed in cubic centimeters per gram. More specifically, the caliper of a single-ply product is measured as the total thickness of the single ply. For multi-ply products, the caliper is measured as the total thickness for the multi-ply product as a whole. In all cases, the caliper is measured on five or six representative product samples and the measurements are averaged. Caliper is measured in accordance with TAPPI test methods T402 “Standard Conditioning and Testing Atmosphere For Paper, Board, Pulp Handsheets and Related Products” and T411 om-89 “Thickness (caliper) of Paper, Paperboard, and Combined Board” with Note 3 for stacked sheets. The micrometer used for carrying out T411 om-89 is an Emveco 200-A Tissue Caliper Tester available from Emveco, Inc., Newberg, Oreg. The micrometer has a load of 2.00 kilo-Pascals (132 grams per square inch), a pressure foot area of 2500 square millimeters, a pressure foot diameter of 56.42 millimeters, a dwell time of 3 seconds and a lowering rate of 0.8 millimeters per second.

As used herein, the “Fuzz-On-Edge” (FOE) value is determined by an image analysis test that measures the fuzziness of a tissue sheet. The test is described in U.S. Pat. No. 6,585,855 B2 entitled “Paper Product Having Improved Fuzz-On-Edge Property”, issued Jul. 1, 2003 to Drew et al., which is hereby incorporated by reference. In general, FOE is determined by bending a tissue sample over a glass slide and viewing it from the side. For a sufficiently large number of samples to be representative, the ratio of the total protruding fiber perimeter length (PR) per sample edge length (EL) is the FOE for the sample. A higher number represents greater protruding fiber lengths on the surface of the web, which enhances the fuzzy perception of the surface by the consumer.

As used herein, “void volume” is a measure of the open space within a sheet as determined by the weight of a non-polar liquid that can be absorbed. The tissue sheets of this invention can have a void volume of from about 11 to about 18 grams per gram of fiber, more specifically from about 11 to about 15 grams per gram of fiber. The measurement of void volume is disclosed in the aforementioned U.S. Pat. No. 6,585,855 B2, previously incorporated by reference.

In the interests of brevity and conciseness, any ranges of values set forth in this specification contemplate all values within the range and are to be construed as written description support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

### BRIEF DESCRIPTION OF THE DRAWINGS

FIG. 1 is a schematic diagram of a crescent former tissue making process useful for purposes of this invention.

FIG. 2 is a schematic diagram of an alternative tissue making process illustrating three different options for the location of the air former for placing the air-laid layer on the air side of the sheet.

FIG. 3 is a schematic diagram of an alternative tissue making process illustrating two different options for the location of the air former for placing the air-laid layer on the dryer side of the sheet.

FIG. 4 is a schematic diagram of the pilot tissue machine process used for making the tissue sheets of Example 1.

FIG. 5 is a cross-sectional photograph of a tissue sheet in accordance with this invention, illustrating the density difference between the wet-laid outer layer and the air-laid outer layer.

### DETAILED DESCRIPTION OF THE DRAWINGS

As used herein, unless otherwise stated, when the same reference number is used in more than one figure, it is intended to represent the same feature.

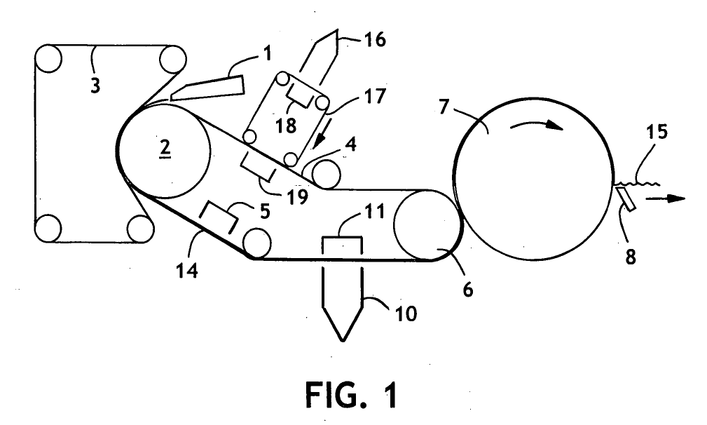

Referring now to FIG. 1, certain aspects of the invention will be described in greater detail. Shown is a wet-laid tissue machine with a crescent former forming section which has been supplemented with an air-laid former in accordance with this invention to provide an air-laid layer of fibers on the dryer side of the sheet. Shown is the headbox 1, the forming roll 2, forming fabric 3, felt or fabric 4, vacuum dewatering box 5, pressure roll 6, Yankee dryer 7 and creping blade 8. Miscellaneous guide rolls for the fabric runs are not labeled. Also shown is the air-laid former 10 and complementary vacuum box 11. In operation, the headbox injects an aqueous suspension of papermaking fibers into the forming zone formed between the forming fabric and a felt, for example. Some water passes through the forming fabric and some water is absorbed by the felt. The resulting wet-laid web 14 adheres to and is supported by the felt, which is partially dewatered by the dewatering vacuum box. At this point in the process, the web contains approximately 75 percent water (25 percent consistency). The web is then passed to the air former 10 which deposits a layer of air-laid fibers directly onto the web. Formation and retention of the air-laid layer is enhanced by the vacuum pulled through the felt by vacuum box 11. At this point the web consistency becomes about 50 percent, assuming the air-laid layer provides approximately 50 percent of the fibers in the web. The resulting layered web is passed between the pressure roll 6 and the Yankee dryer to express additional water from the web and to adhere the web to the Yankee dryer. The dried web is then creped to form a creped sheet 15.

Also shown is an alternative embodiment in which the air-laid web is deposited onto a forming fabric 17 by an air former 16 with the aid of vacuum box 18. The air-laid web is transferred to the felt 4, optionally with the assistance of vacuum box 19, prior to the formation of the wet-laid web, where the wet-laid web and the air-laid web are combined.

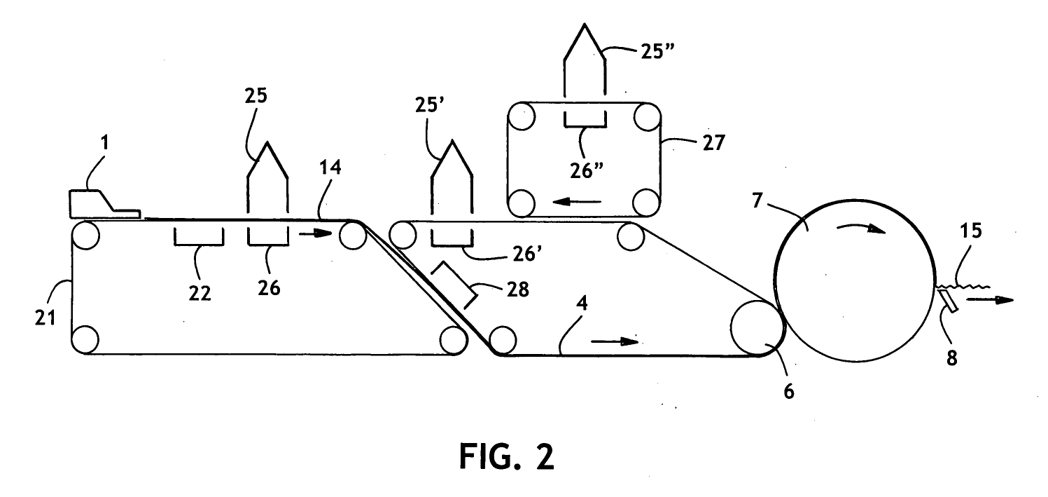

FIG. 2 is a schematic diagram of an alternative process embodiment in accordance with this invention, illustrating three alternative optional locations for an air former that places the air-laid layer of fibers on the felt (or fabric) side of the sheet. In this embodiment, the headbox 1 deposits the fiber suspension onto a forming fabric 21. The resulting wet-laid web 14 is partially dewatered by vacuum box 22. At this point, the air-laid layer of fibers is introduced to the wet-laid web by one of two means. The first is direct addition, as illustrated by the position of air former 25 and vacuum box 26. Alternatively, the air-laid fibers can first be deposited, directly or indirectly, onto the felt 4 and then combined with the wet-laid web. The air former 25′ and vacuum box 26′ are positioned for direct deposition of the air-laid fibers onto the felt. The air former 25″ and vacuum box 26″ are positioned for indirect deposition of the air-laid fibers onto the felt by first depositing the air-laid fibers onto a air-laid forming fabric 27 and thereafter transferring the air-laid web to the felt or fabric. Transfer may be enhanced by vacuum (not shown), if desired.

After the wet-laid and air-laid webs are combined, transfer of the composite web to the felt is assisted by vacuum box 28. The resulting layered web is then transferred to the Yankee dryer at the pressure roll nip, dried and creped.

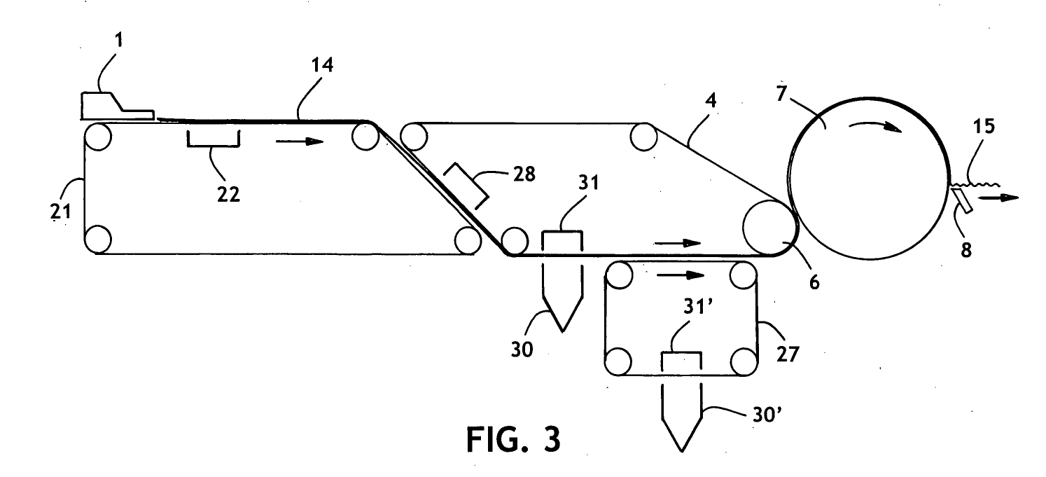

FIG. 3 is a schematic diagram of an alternative process embodiment in accordance with this invention, illustrating two alternative optional locations for an air former that places the air-laid layer of fibers on the dryer side of the sheet. In this embodiment, the headbox 1 deposits the fiber suspension onto a forming fabric 21. The resulting wet-laid web is partially dewatered by vacuum box 22. The web is then transferred to the felt 4 (or fabric) with assistance from vacuum box 28. At this point, the air-laid fibers are deposited onto the wet-laid web, either directly or indirectly. Direct addition is illustrated by the position of air-laid former 30 and vacuum box 31. Indirect addition is illustrated by the position of air-laid former 30′ and vacuum box 31′, where an air-laid web of fibers is first deposited onto an air-laid forming fabric 27 prior to being transferred to the dryer side of the wet-laid web. The resulting layered web is then transferred to the Yankee dryer at the pressure roll nip, dried and creped.

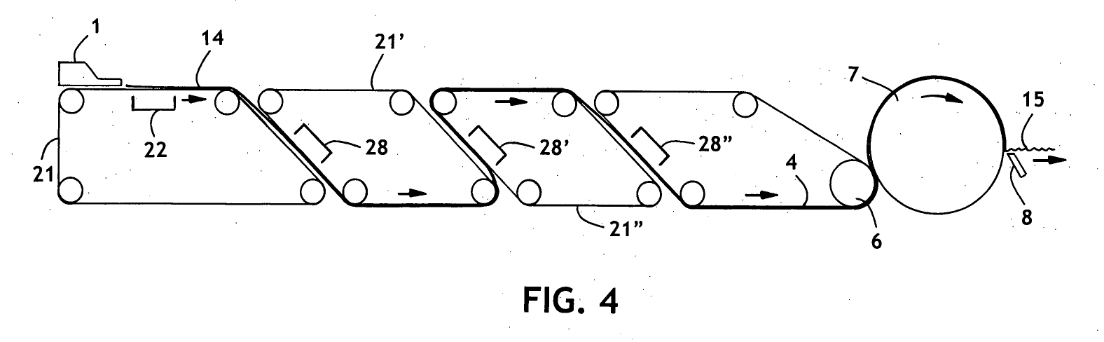

FIG. 4 is a schematic diagram of the process used to produce the sheets of Example 1 described below. In this embodiment, the headbox 1 deposits the fiber suspension onto a forming fabric 21. The resulting wet-laid web 14 is partially dewatered by vacuum box 22. A pre-formed air-laid web is introduced to the wet-laid web prior to the nip between the web supported by fabric 21 and fabric 21′ to position the air-laid web away from the Yankee dryer surface (sample placement 1), or between the web supported by fabric 21′ and fabric 21″ to position the air-laid web next to the Yankee dryer surface (sample placement 2). The combined web is carried to the Yankee dryer by fabric 4. Transfer at the various locations may be enhanced by vacuum boxes 28, 28′ and 28″, if desired.

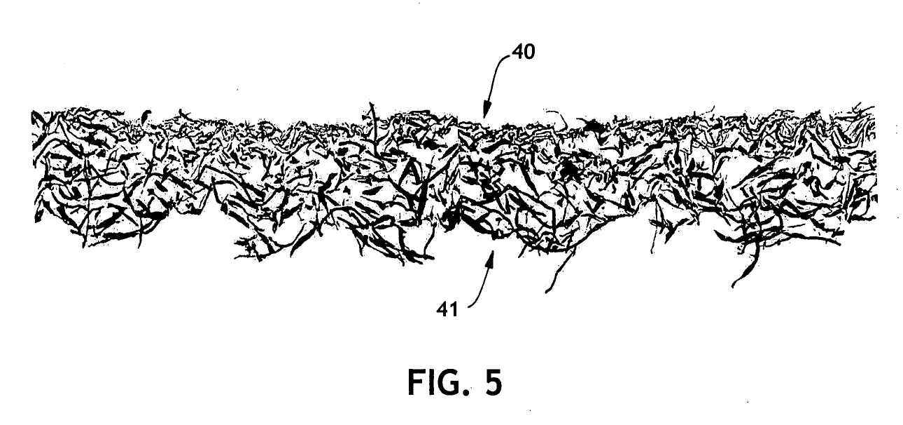

FIG. 5 is a cross-sectional photograph of a tissue sheet in accordance with this invention (Example 1, sample 2 below). Shown are the wet-laid outer layer 40 and the air-laid outer layer 41. As shown, the air-laid outer layer is visibly less dense than the wet-laid outer layer.

### EXAMPLES

### Example 1

In order to further illustrate the invention, air-laid handsheets were made and combined with a wet-laid web on a continuous pilot tissue machine as described in FIG. 4.

More specifically, the air-laid handsheets were produced using two different fiber types. One fiber type was a hardwood fiber and the other was a softwood fiber. The hardwood fiber was derived from an Aaracruz eucalyptus pulp treated with a polysiloxane at an add-on level of 0.7 dry weight percent to improve softness. The coarseness index for the eucalyptus fibers was about 6.8 mg/100 m and the average fiber length was about 0.6 mm. The softwood fiber was a commercially available Scandinavian softwood pulp, Rauma Biobrite™, which contains a debonder. The average fiber length was about 0.9 mm. and the coarseness index was about 13.9 mg/100 m. A Kamas laboratory fiberizer (Type H-01 from Kamas Industries, Vellinge, Sweden) was used to fiberize the pulp in preparation for making handsheets. However, in order to minimize the number of nits in the fiberized pulp, both pulps were pretreated by wet-refining in a TAPPI disintegrator using conventional procedures. Ten pounds per ton of debonder (Witco C-6092) was added to the eucalyptus pulp. The pulp was then formed into a 400 gsm handsheet and dried in preparation for fiberization.

Air-laid handsheets were made using a Buckeye handsheet former. In general, the former consists of an inlet for inserting the pulp, several air inlets, a rotating fan blade and rotating air outlets to break up the pulp into individual fibers, a vacuum source to draw fibers through the former, and a screen-covered outlet. A forming fabric is placed on top of the screen to prevent fibers from passing through the screen into the vacuum. The dimensions of the resulting air-laid handsheets were eight inches by eight inches, which was achieved using a cardboard mask having an eight inches square opening.

After the air-laid handsheets were formed, a binder was sprayed onto the surface of the handsheet to give it integrity. One binder used was a water-soluble cationic acrylate binder, EQs10s, comprising 89.9% mole % ethyl acrylate, 0.1 mole % methyl methacrylate and 10 mole % [2-(methacryloyloxy)ethyl]trimethyl ammonium chloride. The other binder used was a self-crosslinking ethylenevinylacetate latex binder (Airflex EN 1165 available from Air Products, Inc.). The binder application was performed using a spray cabinet which consisted of a pressurized vessel and nozzle which traversed the length of the sample to provide a uniform surface application on the exposed side of the sheet. An add-on of 12.5 dry weight percent was used. After the sheets were sprayed, they were placed in a through-air-dryer to dry the sheet and/or cure the binder.

The resulting air-laid handsheets were then combined with a continuous wet-laid tissue web as described in FIG. 4 above. Specifically, the sheet was formed on a Fourdrinier machine with a single layer headbox. The sheet was a blend of LL19 softwood fibers and Aracruz eucalyptus fibers in a ratio of about 30:70. The machine operated at 50 feet/minute. The sheet was transferred from the forming fabric to a fine mesh transfer fabric with the aid of a vacuum pick-up shoe, transferred to a TAD fabric, then finally to an impression fabric that pressed the sheet to a Yankee dryer. The sheet was dewatered with vacuum boxes, and no other drying was done prior to the Yankee. The sheet was dried to a temperature of 110° C. and then creped. A polyvinyl alcohol based adhesive (CELVOL 523, manufactured by Celanese) was used to adhere the sheet to the Yankee dryer.

When combining the air-laid handsheets with the continuously formed wet-laid web, two different sample placement locations were used. One location was right after the forming section, which enabled the air-laid handsheet (air-laid layer) to be on the felt-side of the resulting tissue sheet. The second location was before the transfer to the first felt, which allows the air-laid handsheet to be on the dryer-side of the resulting tissue sheet. The air-laid handsheet samples were placed on the tissue machine one at a time and collected after the Yankee dryer. Most of the resulting tissue sheets had a basis weight of about 60 gsm and had a very high crepe ratio because the tension was not controlled after the doctor blade. Twenty-five different runs were made during the trial. Many runs did not result in samples useful for further testing because of severe wrinkling of the tissue sheet or because the handsheets fell off of the fabrics prior to the dryer. Of the twenty-five runs, three samples were deemed suitable for further testing, although many of the others may have performed as well or better than the chosen three but were damaged.

Table 1 details the experimental design and sets forth some of the results of the trial, including the bulk of the tissue and whether or not each side of the tissue was creped. Most of the air-laid handsheets that had the binder side facing outwardly attached themselves to the felt and did not make it to the dryer. Many of the handsheets were folded-over or wrinkled when going through the tissue machine. The handsheets that were a combination of an air-laid and a wet-pressed handsheet were very weak coming off of the machine. This could be because of the high percentage of polysiloxane in the wet-pressed basesheet. None of the air-laid handsheet samples containing the eucalyptus fibers made it to the end of the tissue machine.

A key follows the table to better explain the parameters involved. “Handsheet Placement” identifies where the air-laid handsheet was placed on the tissue machine. “Sample Type” describes the type of fiber used in the air-laid sheet as well as if the air-laid sheet was combined with a wet-laid handsheet before being placed on the tissue machine. “Binder Type” gives information on what binder was used, if any. “Sheet Orientation” details the sheet position in relation to the dryer.

TABLE 1
(Experimental Summary for Example 1)

| | | | | Sheet | |
|---|---|---|---|---|---|
| | | Sample | Binder | Orientation | |
| | Handsheet | Type | Type | (A, B, H, W) | Bulk |
| Sample | Placement | (C, D, E, F) | (M, N, O) | dryer --> felt | (cc/g) |
| 1 | 1 | D | N | WA | 9.2 |
| 2 | 1 | D | N | WA | 8.9 |
| 3 | 1 | E | O | WHAB | — |
| 4 | 1 | E | O | WBAH | 7.0 |
| 5 | 1 | C | O | WAB | — |
| 6 | 1 | D | O | WAB | 14.4 |
| 7 | 1 | D | O | WBA | 7.6 |
| 8 | 1 | D | O | WBA | 7.5 |
| 9 | 1 | E | M | WHAB | 10.6 |
| 10 | 1 | E | M | WBAH | 6.9 |
| 11 | 1 | D | M | WAB | — |
| 12 | 1 | D | M | WBA | 7.8 |
| 13 | 1 | F | N | WHA | 11.7 |
| 14 | 1 | F | N | WAH | 12.1 |
| 15 | 1 | F | N | WHA | 10.4 |
| 16 | 1 | F | N | WAH | 12.1 |
| 17 | 2 | D | N | AW | 8.8 |
| 18 | 2 | E | O | BAHW | — |
| 19 | 2 | C | O | BAW | — |
| 20 | 2 | D | O | ABW | 8.1 |
| 21 | 2 | D | O | BAW | 9.2 |
| 22 | 2 | D | O | ABW | 8.4 |
| 23 | 2 | E | M | HABW | 8.2 |
| 24 | 2 | D | M | ABW | 10.4 |
| 25 | 2 | D | M | BAW | 10.6 |

Key:
Handsheet placement
1 On the forming section, after the vacuum box.
2 Just before the second transfer.
Sample type
C Air-laid sheet with 70/30 ratio of (eucalyptus with 0.5% debonder and 0.7% polysiloxane add- on) and (thermobond fibers); heat-treated at 240° F. for 2 minutes under 80 psi
D Air-laid sheet with 75/25 ratio of (air-laid, wet-refined Biobrite fibers) and (thermobond fibers); heat-treated at 240° F. for 1.5 minutes under 160 psi
E Sheet of type “D” above formed on a handsheet made with eucalyptus pulp that had 0.7% polysiloxane added
F Sheet of type “D” above couched to a wet handsheet made with eucalyptus pulp that had 0.7% polysiloxane added
Binder type
N No binder
M Airflex EN 1165
O EQs10S
Sheet orientation - The resultant layer structure of each sample, the orientation noted with code letters reading from left to right, corresponding to the dryer side to felt side (also referred to as the air side). Thus, a basesheet structure labeled “WHA” has the wet-laid tissue layer next to the Yankee dryer, a wet-laid handsheet next to it, and an air-laid handsheet farthes from the dryer (the felt, or air, side)
A Air-laid handsheet
B Binder (indicates the bonded side of the air laid sheet)
H Wet-laid handsheet
W Wet-laid tissue basesheet (blended eucalyptus/LL19 softwood) produced on the tissue machine

Of the samples in which the handsheet layer made it through the process, some interesting properties were noted. The sheets were very “two-sided” with the air-laid side being significantly fuzzier. The profile of fibers through the sheet appeared to be very different for the air-laid and wet-laid halves. Three samples (sample numbers 2, 7 and 20) were tested for FOE and z-directional profile analysis to verify their unique properties. It was also discovered that the bulks of the combination sheets of this invention were higher than those of traditional facial tissue sheets. Bulk values of 7-8 cc/g can be expected from typical two-ply facial tissues, while bulk values of 7-13 cc/g were measured for the experimental tissues. Though the increase may not seem significant, the experimental tissue sheets have a higher basis weight and are only single-ply sheets, for which it is more difficult to attain a high bulk than for two-ply products.

The FOE data for samples 2, 7, 20 are listed in Table 2 below. The data show extremely high FOE values on the air-laid side and low FOE values on the wet-laid side for samples 2 and 7. Sample 20 exhibits a high FOE value on both sides, but the sample was not well-creped. These FOE values are much higher than those exhibited by typical tissues.

TABLE 2
(FOE Values for Example 1)

| Sample | FOE (Air-laid side) | FOE (Wet-laid side) |
|---|---|---|
| 2 | 5.75 | 0.47 |
| 7 | 3.43 | 0.78 |
| 20 | 2.25 | 2.55 |

### Example 2

Trials were conducted in the configuration of FIG. 2 using a Weavex Millenium 1C felt as the fabric 4. A blended wet-laid basesheet of 50 percent Aracruz eucalyptus hardwood and 50 percent northern softwood kraft (LL19) was made with headbox 1 and partially dewatered with vacuum. An air former (25) was placed over a vacuum slot (26). The air former was supplied with a dilute mixture of dry fiber and air by feeding strips (2 inches×18 inches) of Rauma Biobrite pulp into the feeder slot of the Kamas H-01 Laboratory Hammermill fiberizer at a constant rate so as to deliver a continuous flow of fiberized fluff through a rubber hose and into the forming box (25). The fiber was transported with the aid of an EXAIR air amplifier mounted in-line with the hose and supplied with compressed air for operation. Air flows were adjusted to maintain a velocity of about 20 meters per second through the hose to minimize fiber deposition or clumping in the hose. Baffles inside the forming box (25) aided in distributing the fibers uniformly across the wet-laid tissue web. The air-laid fibers were drawn toward the vacuum slot and bonded to the wet-laid tissue well enough to transfer successfully from the forming fabric to the felt. The sheet then continued on to the Yankee dryer and past the creping blade. The sheet was dried to a temperature of 110° C. Because the sheet transfer and creping were successful, it was deemed unnecessary to add any binder chemicals. However, binder chemicals could have been added either in the forming box or immediately after it.

Several different basis weights, mixtures, and product forms were made, as outlined in Table 3 below. Comparisons were made for both softroll tissue (directly from the tissue machine) and finished product that was lightly calendered and, in some cases, converted into a 2-ply tissue product. The 2-ply product was plied with the wet-laid side (layer) of each ply facing outward.

TABLE 3
(Experimental Summary for Example 2)

| | Basis Weight (gsm) | | | | | | |
|---|---|---|---|---|---|---|---|
| | Air- | Wet- | At | Finished | # of | Speed | |
| Code | laid | laid | Dryer | Product | Plies | (ft/min) | Description |
| 0 | 0 | 12 | 15 | 29.6 | 2 | 50 | Standard 2-ply wet-laid tissue used as |
| | | | | | | | control code. |
| 1 | 6 | 12 | 20 | 22.0 | 1 | 50 | 1-ply product with air-laid fibers on the |
| | | | | | | | felt side, wet-laid fibers on the dryer |
| | | | | | | | side. |
| 2 | 6 | 12 | 20 | 41.1 | 2 | 50 | 2-ply product with air-laid fibers facing |
| | | | | | | | each other (inward), dryer (wet-laid) |
| | | | | | | | side facing out. |
| 3 | 12 | 12 | 30 | 64.3 | 2 | 50 | Heavy basis weight version of code 2. |
| 4 | 4 | 9 | 16 | 31.7 | 2 | 75 | Target basis weight 2-ply sheet with air- |
| | | | | | | | laid fibers facing each other, dryer (wet- |
| | | | | | | | laid) side facing out. |

The most notable properties of these codes are high fuzz-on-edge (FOE) values on the air-laid side, high void volume and increased bulk due to higher calipers. FOE values for the air-laid side of code 1 ranged as high as 3.4, compared to 0.7 for the control (code 0). Caliper for code 4 was 259 μm, compared to 205 μm for the control. Consequently, bulk was increased nearly 20% from the control (6.9 cc/g) to code 4 (8.2 cc/g). Additionally, the void volume values were very high, 14.3 for code 4 compared to 10.3 for the control. The results are tabulated in Table 4 below. Note that calipers were always measured on two plies except for code 1, which was treated as a 1-ply product. The designation “FP” refers to finished product and the designation “TM” refers to the soft roll after the tissue machine, but prior to any converting.

TABLE 4
(Experimental Data for Example 2)

| | Basis | | | | | |
|---|---|---|---|---|---|---|
| | Weight | Caliper | Bulk | Void | FOE | FOE |
| Code | (gsm) | (μm) | (cc/g) | Volume | (Air Side) | (Wet Side) |
| 0 FP | 29.6 | 205 | 6.9 | 10.3 | | 0.65 |
| 1 FP | 22.0 | 162 | 7.4 | 11.3 | 2.87 | 0.76 |
| 2 FP | 41.1 | 299 | 7.3 | 11.4 | | |
| 3 FP | 64.3 | 450 | 7.0 | 11.5 | | |
| 4 FP | 31.7 | 259 | 8.2 | 14.3 | | 0.49 |
| 0 TM | 32 | 275 | 8.6 | | | 0.55 |
| 1 TM | 23 | 196 | 8.5 | | 3.14 | 0.33 |
| 2 TM | 46 | 445 | 9.7 | | | |
| 3 TM | 62 | 528 | 8.5 | | | |
| 4 TM | 34 | 336 | 9.9 | | 0.65 | |

It will be appreciated that the foregoing description and examples, given for purposes of illustration, are not to be construed as limiting the scope of this invention, which is defined by the following claims and all equivalents thereto.

1. A unitary layered tissue sheet having two outer surfaces, said sheet comprising: (a) an air-laid outer layer of papermaking fibers which provides one outer surface of the sheet; and (b) a wet-laid outer layer of papermaking fibers which provides the second outer surface of the sheet, wherein the air-laid outer layer is relatively fuzzy compared to the wet-laid outer surface.

2. A multi-ply tissue product having two outer plies, wherein each of the two outer plies is a tissue sheet having two outer surfaces, said sheet comprising: (a) an air-laid outer layer of papermaking fibers which provides one outer surface of the sheet; and (b) a wet-laid outer layer of papermaking fibers which provides the second outer surface of the sheet, wherein the air-laid outer layer is relatively fuzzy compared to the wet-laid outer surface.

3. The tissue product of claim 2 wherein the two outer plies are oriented such that both air-laid outer surfaces are outwardly-facing.

4. The tissue product of claim 2 wherein the two outer plies are oriented such that both air-laid surfaces are inwardly-facing.

5. The tissue product of claim 2 wherein the two outer plies are oriented such that one air-laid surface is outwardly-facing and the other air-laid surface is inwardly-facing.

6. The tissue sheet of claim 1 or 2 wherein the air-laid outer layer consists primarily of softwood fibers.

7. The tissue sheet of claim 1 or 2 wherein the air-laid outer layer comprises softwood fibers and thermally bonded synthetic fibers.

8. The tissue sheet of claim 1 or 2 wherein the air-laid outer layer comprises softwood fibers and a chemical binder.

9. The tissue sheet of claim 1 or 2 wherein the Fuzz On Edge (FOE) value of the air-laid outer surface of the sheet is greater than the FOE value of the wet-laid outer surface of the sheet such that the difference in FOE values between the two outer surfaces is about 2.00 or greater.

10. The tissue sheet of claim 1 or 2 wherein the Fuzz On Edge (FOE) value of the air-laid outer surface of the sheet is greater than the FOE value of the wet-laid outer surface of the sheet such that the difference in FOE values between the two outer surfaces is about 2.50 or greater.

11. The tissue sheet of claim 1 or 2 wherein the Fuzz On Edge (FOE) value of the air-laid outer surface of the sheet is greater than the FOE value of the wet-laid outer surface of the sheet such that the difference in FOE values between the two outer surfaces is about 3.00 or greater.

12. The tissue sheet of claim 1 or 2 wherein the Fuzz On Edge (FOE) value of the air-laid outer surface of the sheet is greater than the FOE value of the wet-laid outer surface of the sheet such that the difference in FOE values between the two outer surfaces is from about 2.00 to about 6.00.

13. The tissue sheet of claim 1 or 2 wherein the Fuzz On Edge (FOE) value of the air-laid outer surface of the sheet is greater than the FOE value of the wet-laid outer surface of the sheet such that the difference in FOE values between the two outer surfaces is from about 2.50 to about 5.00.

14. The tissue sheet of claim 1 or 2 wherein the Fuzz On Edge (FOE) value of the air-laid outer surface of the sheet is greater than the FOE value of the wet-laid outer surface of the sheet such that the difference in FOE values between the two outer surfaces is from about 3.00 to about 4.00.

15. The tissue sheet of claim 1 or 2 having a sheet bulk of from about 7 to about 15 cubic centimeters per gram.

16. The tissue sheet of claim 1 or 2 having a sheet bulk of from about 9 to about 15 cubic centimeters per gram.

17. The tissue sheet of claim 1 or 2 having a sheet bulk of from about 10 to about 14 cubic centimeters per gram.

18. The tissue sheet of claim 1 or 2 having a void volume of from about 11 to about 18 grams per gram.

19. The tissue sheet of claim 1 or 2 having a void volume of from about 11 to about 15 grams per gram.

20. The tissue sheet of claim 1 or 2 wherein the air-laid outer layer is relatively less dense than the wet-laid outer layer.

21. A multi-ply tissue product having two outer plies, wherein each of the two outer plies is a tissue sheet having two outer surfaces, said sheet comprising: (a) an air-laid outer layer of papermaking fibers which provides one outer surface of the sheet; and (b) a wet-laid outer layer of papermaking fibers which provides the second outer surface of the sheet, wherein the air-laid outer layer is relatively less dense than the wet-laid outer layer, wherein the Fuzz On Edge (FOE) value of the air-laid outer surface of the sheet is greater than the FOE value of the wet-laid outer surface of the sheet and such that the difference in FOE values between the two outer surfaces is from about 2.00 to about 6.00, said product having a bulk of from about 10 to about 14 cubic centimeters per gram.

22. A method of making a unitary layered tissue sheet comprising:
(a) forming a wet-laid web by depositing an aqueous suspension of papermaking fibers onto a forming fabric to form a wet web;
(b) forming an air-laid web by depositing an airborne suspension of papermaking fibers onto a forming fabric;
(c) combining the air-laid web with the wet web to form a layered web having a wet-laid layer and an air-laid layer; and
(d) drying the layered web.

23. The method of claim 22 wherein the dried web is creped.

24. The method of claim 22 wherein the layered web is dried and creped by adhering the wet-laid layer side of the web to the surface of a Yankee dryer.

25. The method of claim 22 wherein the wet-laid web is formed on a crescent former, in which the aqueous suspension of papermaking fibers is injected into a forming zone created by the convergence of a forming fabric and a felt, wherein the air-laid web is transferred from the air-laying forming fabric to the felt prior to the formation of the wet-laid web.

26. The method of claim 22 wherein the air-laid web is combined with the wet web after the wet web is formed.

27. A method of modifying a wet-press tissue machine comprising a headbox for depositing an aqueous suspension of papermaking fibers onto a forming fabric to form a wet-laid web, a drying section for drying the wet-laid web, and a transfer section in which the wet-laid web is supported by a fabric or felt and transferred to the drying section, wherein the tissue machine is modified by adding an air former for depositing an air-laid web of papermaking fibers and positioning the air former such that the air-laid web of fibers is combined with the wet-laid web prior to the drying section.

28. The method of claim 27 wherein the air former is positioned-to deposit the air-laid web of fibers onto the wet-laid web while the wet-laid web is supported by a forming fabric.

29. The method of claim 27 wherein the transfer section of the tissue machine comprises a transfer fabric or felt to which the wet-laid web is transferred from the forming fabric, wherein the air former is positioned to deposit the air-laid web of fibers onto the transfer fabric or felt.

30. The method of claim 27 wherein the tissue machine comprises a crescent former wherein the wet-laid web is formed between a forming fabric and a felt and carried to the drying section by the felt, wherein the air former is positioned to deposit the air-laid web of fibers onto the felt.
